(19) United States
(12) Patent Application Publication
(10) Pub. No.: US 2009/0186366 A1
(43) Pub. Date: Jul. 23, 2009

(54) METHOD OF DETECTING INTERACTIONS BETWEEN PROTEIN COMPONENTS

(75) Inventors: Sofia Edlund, Uppsala (SE); &Aring;sa Hagner-McWhirter, Uppsala (SE); Henrik Neu, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 12/300,792

(22) PCT Filed: Jun. 8, 2007
(86) PCT No.: PCT/SE07/00560

§ 371 Date: Nov. 14, 2008

(30) Foreign Application Priority Data
Jun. 28, 2006 (SE) 0601424-5

(51) Int. Cl. G01N 33/53

(52) U.S. Cl. 435/7.1

(57) ABSTRACT

A method of detecting protein interactions in a biological sample. Protein components in a first aliquot of a sample are labelled with a first bifunctional dye which cross-links any-interacting components. A second aliquot of said sample functions as a control sample, in which protein components are labelled with a different, mono functional dye. Thereafter, the two aliquots are mixed and all components are separated by electrophoresis. Finally, differences in luminescence of the separated dye-labelled components are detected. The two dyes should match one another with regard to charge and/or molecular weight but should emit different kinds of fluorescent light.

### CROSS-REFERENCE TO RELATED APPLICATIONS

This application is a filing under 35 U.S.C. § 371 and claims priority to international patent application number PCT/SE2007/000560 filed Jun. 8, 2007, published on Jan. 3, 2008, as WO 2008/002236, which claims priority to patent application number 0601424-5 filed in Sweden on Jun. 28, 2006.

### FIELD OF THE INVENTION

The present invention relates to a method for the detection of interactions between components in a sample, in particular protein-protein interactions in cellular samples.

### BACKGROUND OF THE INVENTION

Protein-protein interactions are important elements for the understanding of cellular processes, operating at virtually every level of cell function, for example regulation of gene expression, transport, signal transduction and cell cycle control. A major contributor towards an understanding of protein functionality is the identification of protein-protein interactions and interacting protein partners. Many human diseases are the result of abnormal protein-protein interactions involving endogenous proteins, proteins from pathogens or both. Several recent studies have identified and/or characterised specific interactions from various disease systems, including cervical cancer, bacterial infection, leukaemia and neurodegenerative disease such as Creutzfeld-Jacob and Alzheimer's disease (see for example, Ryan, D. P, and Matthews, J. M., Protein-protein interactions in human disease, Curr. Opin. Struct. Biol., (2005), 15, 441-6). The inhibition of aberrant protein-protein associations is of obvious clinical importance; however, because of the diverse nature of protein-protein interactions, the successful design of therapeutics requires a detailed knowledge of each system at a molecular level. Furthermore, a number of approaches are being made to identify and characterise inhibitors of protein-protein interactions that may form useful therapeutics for human disease.

A number of different approaches to the study of protein-protein interactions have been reported, including size exclusion chromatography, protein affinity chromatography, LC-MS analysis, fluorescence (FRET) spectroscopy and the two-hybrid system. (For a review, see Phizicky, E. and Fields, S., Protein-protein Interactions: Methods for Detection and Analysis, Microbiological Reviews, (1995), 59(1), 94-123). One such technique employs chemical cross-linking in a method to characterise protein-protein interactions, firstly to deduce protein architecture or assembly of proteins, and secondly to detect protein-protein interactions, both in vivo and in vitro. For example, detection of interacting proteins may be accomplished in vivo by the use of membrane permeable cross-linking reagents followed by immuno-precipitation of the cross-linked protein complex (de Gunzberg, J. et al, Proc. Nat. Acad. Sci., (1989), 86, 4007-11). Detection of interacting proteins has been achieved in vitro by methods involving proteins labelled with iodine-125 or sulphur-35 (Sarkar, F, et al, Proc. Nat. Acad. Sci., (1984), 81, 5160-64; Mita, S. et al, Proc. Nat. Acad. Sci., (1989), 86, 2311-15), by the use of labelled cross-linking reagents (Denny, J. B. et al, Proc. Nat. Acad. Sci., (1981), 81, 5286-90), or by cross-linking coupled with mass-spectroscopy (Sinz, A. J Mass Spec. (2003), 38, 1225-37; Bernhard, O. et al, Biochemistry (2004), 43, 256-64).

Proteins can be separated with a high degree of resolution by means of one-dimensional (1D) electrophoresis or two-dimensional (2D) electrophoresis. 1D electrophoresis is a standard separation technique in which proteins are separated by differential migration along one axis of a separation medium, such as a polyacrylamide gel. In 2D electrophoresis, proteins are separated according to their respective isoelectric points in a first dimension by the now well known technique of isoelectric focusing and by molecular weight in the second dimension by discontinuous SDS electrophoresis (O'Farrell, P. H. J. Biol. Chem., (1975), 250, 4007-4021). Two-dimensional polyacrylamide gel electrophoresis (2D PAGE) is a more sensitive method of separation and will provide resolution of most of the proteins in a sample. Proteins migrate in one- or two-dimensional gels as bands or spots, respectively. The separated proteins are visualized by a variety of methods; by staining with a protein specific dye, by protein mediated silver precipitation, autoradiographic detection of radioactively labeled protein, or by covalent attachment of fluorescent compounds.

WO 96/33406 (Minden, J. and Waggoner, A. S.) describes a method whereby different cell samples are lysed and the total cellular proteins extracted. The different protein samples are then labelled with dyes that are matched for molecular mass and charge to give equivalent migration in 2D electrophoresis. The approach employs cyanine dyes having an N-hydroxysuccinimidyl (NHS) ester reactive group to label amines. The fluorescent pre-labelling of protein samples allows multiple samples to be run on the same gel, thereby enabling quantitative differences between the samples to be easily identified by overlaying the fluorescent images. However, this method is not able to distinguish interacting proteins from non-interacting proteins and hence is not able to detect protein-protein interaction in biological samples.

### SUMMARY OF THE INVENTION

The present invention provides a method for detecting interactions between components in a cellular or sub-cellular environment, in particular protein-protein interactions, by labelling components with matched fluorescent dyes so as to cross-link interacting components, and thereafter performing an analysis of the labelled components by comparison with non cross-linked components. The method of the invention is particularly useful for detecting protein-protein interactions in a cellular or sub-cellular environment, wherein all interactions between proteins are detected at the same time. According to the present method, false positives are efficiently excluded through the use of an internal control sample, co-migration of test and control samples followed by analysis of overlaid images.

Accordingly, in a first aspect of the invention, there is provided a method of detecting intermolecular association between two components present in a sample, the method comprising:

a) contacting a first aliquot from said sample with a bifunctional reactive moiety under conditions so as to covalently bind to and thereby cross-link said components and wherein said bifunctional reactive moiety comprises a first dye chosen from a matched set of dyes and wherein each dye in said matched set emits luminescent light having a property that is distinguishably different from the emitted luminescent light of the remaining dyes in said matched set;
b) preparing an extract of dye-labelled components from said first aliquot;
c) separating the different dye-labelled components by an electrophoretic method; and
d) detecting differences in a luminescence property between the separated dye-labelled components by luminescence detection;
wherein said separating step c) is performed in the presence of a control comprising an extract of components from a second aliquot from said sample and wherein said second aliquot is contacted with a different dye chosen from said matched set of dyes so as to covalently bind to said components.

In a second aspect, the invention provides a method of detecting intermolecular association between two components present in a sample, said method comprising:

a) contacting a first aliquot from said sample with a bifunctional reactive moiety under conditions so as to covalently bind to and cross-link said components;
b) preparing an extract of components from said first aliquot;
c) contacting said extract with a dye chosen from a matched set of dyes wherein each dye in said matched set is capable of selectively labelling said components and wherein each dye emits luminescent light having a property that is distinguishably different from the emitted luminescent light of the remaining dyes in said matched set;
d) separating the different dye-labelled components by an electrophoretic method; and
e) detecting differences in a luminescence property between the separated dye-labelled components by luminescence detection;
wherein said separating step d) is performed in the presence of a control comprising an extract of components from a second aliquot from said sample and wherein said second aliquot is contacted with a different dye chosen from said matched set of dyes so as to covalently bind to said components.

Preferably, the methods according to the first and second aspects are suitable for detecting intermolecular association between two protein components present in a sample.

Preferably, the sample is a cell sample, including intact cells, tissue samples, and microsomal or other sub-cellular fractions including golgi, mitochondria, chloroplasts and nuclear fractions of cells.

### BRIEF DESCRIPTION OF THE DRAWINGS

FIG. 1a and FIG. 1b are flow diagrams which illustrate the test sample (FIG. 1a) and control sample (FIG. 1b) preparation procedure according to one aspect of the invention.

FIG. 2a and FIG. 2b are flow diagrams illustrating preparation of the test (FIG. 2a) and control (FIG. 2b) in an alternative aspect of the invention.

FIG. 3 is a schematic diagram showing a typical spot pattern obtained in the first aspect of the invention.

FIG. 4 is a schematic diagram showing a typical spot pattern obtained in the second aspect of the invention.

FIG. 5 (Panels A and B) shows images of a mixture of anti-actin antibody and actin (A) and anti-transferrin antibody and transferrin (B), labelled either with mono-reactive CY™3 (control) or with two different concentrations of bis-reactive CY™5. Lane 1 contains only CY™3 labelled control sample whereas lanes 2 and 3 contain a mix of test and control sample.

### DETAILED DESCRIPTION OF THE INVENTION

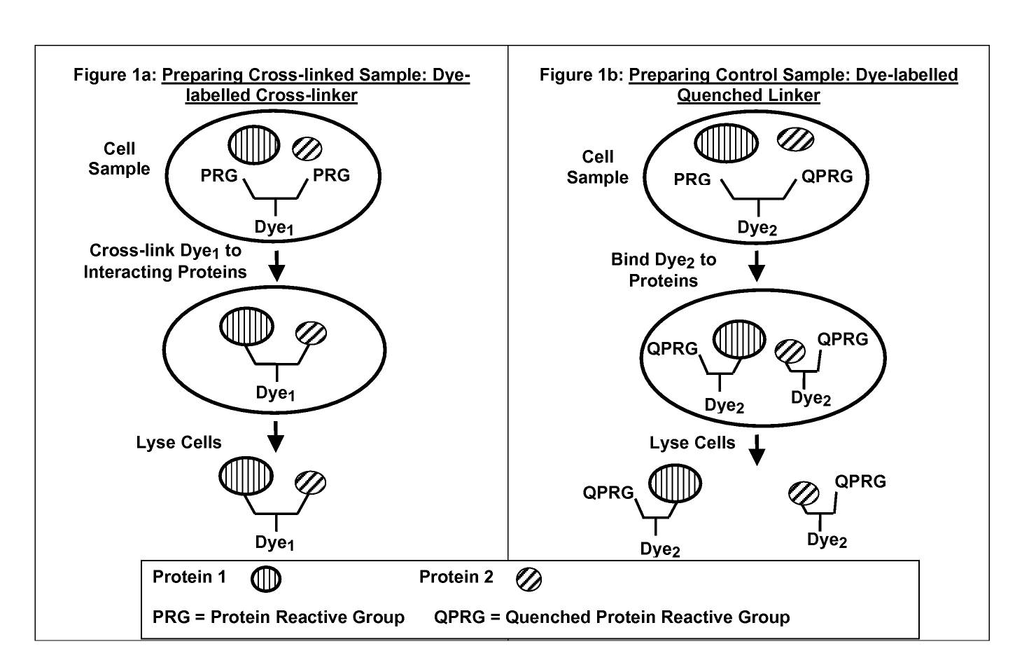

In one aspect as shown in FIG. 1a, the present invention provides in a preferred embodiment, a method to detect protein-protein interactions in a cell sample by separation of interacting or neighbouring proteins from non-interacting or non-neighbouring proteins using an electrophoretic method. The method comprises contacting a first aliquot from the sample (a test sample) with a bifunctional protein reactive moiety (or group), suitably a cross-linking reagent, under conditions so as to covalently bind to and cross-link neighbouring or interacting protein components present in the sample. In this aspect of the invention, the bifunctional reactive moiety comprises a first dye, preferably a bis-reactive fluorescent dye carrying two reactive groups on each dye molecule. As shown in FIG. 1b, a control sample is prepared by treating a second aliquot of the cell sample with a monofunctional reactive moiety (or group) under conditions so as to covalently bind with said protein components, wherein the monofunctional reactive group comprises a second dye. Suitably, the monofunctional reactive moiety may be a partially quenched cross-linking group, or preferably a mono-reactive fluorescent dye that carries only one reactive group. Preferably the sample is a cellular sample. For studying protein-protein interactions in cellular samples, the cross-linking reagent and monofunctional reactive dye should be cell permeable, thereby enabling the reagent to easily enter the cells.

The first aliquot (test sample) and second aliquot (control sample) are each contacted separately with a lysis reagent and the lysed aliquots mixed together to form a mixture. In the alternative, the test and control samples may be mixed before subjecting the mixture to lysing conditions. The dye-labelled protein components in the mixture are separated and differences in the separated dye-labelled components are detected by luminescence detection. Suitably, the first and second dyes are chosen from a matched set of dyes and wherein each dye within said matched set emits luminescent light having a property that is distinguishably different from the emitted luminescent light of the remaining dyes in said matched set. Suitably, the monofunctional reagent may be a cross-linking reagent wherein one of the reactive groups of the reagent is quenched (as shown in FIG. 1b). In the alternative, the monofunctional reagent may be a mono-reactive dye, preferably a fluorescent dye.

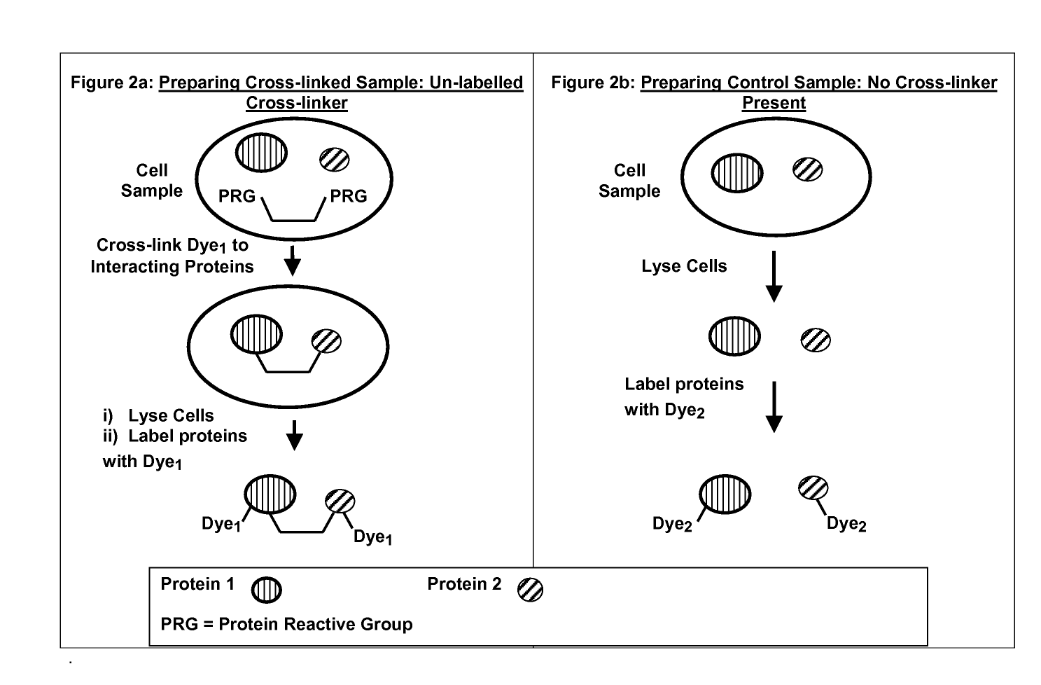

In a second aspect, shown in FIG. 2a, there is provided in a preferred embodiment, a method of detecting intermolecular association between protein components present in a sample. The method comprises contacting a first aliquot from the sample with a bifunctional reactive moiety under conditions so as to cross-link the interacting protein components. The first aliquot, containing cross-linked interacting proteins, is treated with a lysis reagent so as to provide a lysed aliquot. As shown in FIG. 2b, a second aliquot of the cell sample serves as a control sample and is also treated with a lysis reagent to provide a lysed control sample. The first and second lysed aliquots are each contacted with a different luminescent dye chosen from a set of matched luminescent dyes under conditions so as to covalently bind the dye to the protein components in the aliquots. Preferably, each of the dyes in the matched set of dyes is a mono-reactive fluorescent dye capable of covalently binding to protein components in the sample and each dye within said matched set emits luminescent light having a property that is distinguishably different from the emitted luminescent light of the remaining dyes in said matched set. Following binding of the dyes to the protein components, the first and second lysed aliquots are mixed together to form a mixture. Different dye-labelled protein components within the mixture are separated by an electrophoretic method and differences in the separated dye-labelled components are detected by luminescence detection.

Suitably, the first and second dyes are selected from a matched set of dyes, wherein each dye is matched one with the other by virtue of its charge and molecular weight characteristics, such that relative electrophoretic migration of a protein component labeled with any one of the said dyes is the same as relative electrophoretic migration of said component labeled with another dye in the matched set. Thus, the dyes are preferably of approximately equal molecular weight, but need not be. Furthermore, the dyes are matched by charge, such that differentially labelled proteins migrate to same position following separation by electrophoresis in one or two dimensions. Dyes that retain the native charge of the protein are particularly preferred, since there is no pl shift resulting from proteins labelled with such dyes. When lysine is target group of the protein, the dye should possess a net charge of +1; for dyes that target cysteine residues in a protein, the net charge should be neutral. However, the dye-labelled components from the test sample will possess a luminescent property that is different from the luminescent properties of the control sample. Suitably, each of the dyes in the matched set is a fluorescent dye such that each dye emits luminescent light at a wavelength that is sufficiently different from the emitted luminescent light of the remaining dyes in said matched set to provide a detectably different fluorescence signal.

Suitably, the bifunctional reactive moiety contains two reactive terminal groups capable of specifically covalently binding to and cross-linking two (or more) interacting proteins through a covalent linkage. The different terminal reactive groups are specific for different functional groups, for example amino, hydroxyl, sulphydryl and carboxyl groups, which are present on the protein (or other cellular component) to be studied. Cross-linking reagents may be classified generally into two types: i) hetero-bifunctional cross-linking reagents, i.e. those wherein the two reactive terminal groups have dissimilar chemistry, thereby enabling the formation of covalent linkages between unlike functional groups on components to be studied; and ii) homo-bifunctional cross-linking reagents. In a preferred embodiment, the bifunctional reactive moiety is a homo-bifunctional cross-linking reagent, i.e. a reagent having two identical reactive groups chosen to couple like functional groups present on the component, for example the ε-amino groups of lysine residues present in proteins. Alternatively, the homo-bifunctional reactive moiety may covalently bind to and link hydroxyl groups, or sulphydryl groups or carboxyl groups present in the protein component. Suitable reactive terminal groups of the bifunctional reactive moiety may be selected from succinimidyl ester, sulpho-succinimidyl ester, maleimide and hydrazide. Preferably, the reactive group is a succinimidyl ester of a carboxylic acid, or a maleimide.

Suitably, the bifunctional reactive dye cross-linkers and certain monofunctional dyes for use according to the present invention are designed to be cell permeable, such that they penetrate the cellular membrane and thereby label internal cellular proteins. Membrane permeant compounds can be generated by designing dyes and/or cross-linkers having substituents that reduce water solubility, for example lipid and hydrocarbon solubilising groups such as alkyl, aryl and aralkyl groups. Alternatively, hydrophilic groups on the dye chromophore may be masked to provide more hydrophobic compounds. Masking groups may be designed to be cleaved from the cross-linking moiety or the dye by intracellular enzymes to generate the derived substrate intracellularly. Because the cross-linking moiety is more hydrophilic than the membrane permeant derivative, it is then trapped in the cell. Suitable cell membrane permeabilising groups may be selected from acetoxymethyl ester, which is readily cleaved by endogenous mammalian intracellular esterases (Jansen, A. B. A. and Russell, T. J., J.Chem. Soc. (1965), 2127-2132 and Daehne, W. et al, J. Med. Chem. (1970), 13, 697-612 and pivaloyl ester (Madhu et al, J. Ocul. Pharmacol. Ther. (1998), 14, 5, pp 389-399), although other suitable groups including delivery molecules (such as delivery peptides) will be recognised by those skilled in the art.

Suitably, each of the dyes in the matched set should be easily detectable and resolvable by the detection means. Thus, each dye emits luminescent light having a property that is distinguishably different from the emitted luminescent light of the remaining dyes in the set. Preferably, each of the dyes is distinguishably one from the other by virtue of its fluorescence wavelength and/or its fluorescence lifetime. Thus, the luminescent property may be the emission wavelength of the dye, each dye being characterised by its different fluorescence emission wavelength from any other dye in the matched set. Alternatively, the luminescent property of the dyes may be their fluorescence lifetimes, each dye in the set being characterised by a different fluorescence lifetime.

Dyes that possess suitable characteristics for use in the present invention may be selected from the well known classes of fluorescent dyes including fluoresceins, rhodamines, cyanine dyes and derivatives of the bis-pyrromethine boron difluoride dyes.

A preferred class of dye for use in the present invention are the cyanine dyes (see for example, U.S. Pat. No. 6,048,982, Waggoner, A. S.). The cyanine dyes are characterised by strong spectral absorption bands with the absorption being tuneable over a large spectral range by synthetic design. Particularly preferred dyes are selected from cyanine dyes having the structure of formula (I):

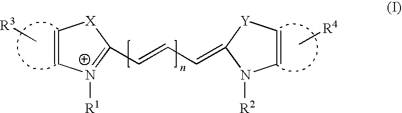

wherein the dotted lines each represent carbon atoms necessary for the formation of said dye and are selected independently from phenyl and naphthyl;
X and Y are the same or different and are selected from: >C(CH₃)₂, O and S;
at least one of groups R¹, R², R³ and R⁴ is the group -E-F where E is a spacer group having a chain from 1-20 linked atoms selected from linear or branched C₁₋₂₀ alkyl chains, which may optionally contain one or more linkages selected from —O—, —NR′—, —C(O)—NR′—, —CR′═CR′— and phenyl, where R′ is hydrogen or C₁-C₄ alkyl, and F is a reactive group capable of reacting with a complementary functional group on the protein component to be labelled;
any remaining group R¹ and R² is selected from C₁-C₁₀ alkyl; and
any remaining R³ and R⁴ are selected from hydrogen, sulphonate and sulphonic acid; and n is an integer from 1 to 3.

In a preferred embodiment, R¹ and/or R² is the group -E-F where E and F are hereinbefore defined, and R³ and R⁴ are hydrogen.

Additional cyanines for use in the method of the present invention are those disclosed in PCT patent application No. WO 2004/085539 (Chen, Chung-Yuan and Kumar, Shiv) entitled “Cyanine Dye Labelling Reagents with Meso-substitution” and having the following general formula (II):

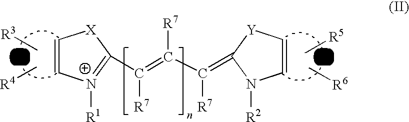

in which groups R³ and R⁴ are attached to the Z¹ ring structure and groups R⁵ and R⁶ are attached to the Z² ring structure, and n=1, 2 or 3; Z¹ and Z² independently represent the atoms necessary to complete one ring, or two fused ring aromatic or heteroaromatic systems, each ring having five or six atoms selected from carbon atoms and optionally no more than two atoms selected from oxygen, nitrogen and sulphur; X and Y are the same or different and are selected from oxygen, sulphur, —CH═CH— and the group:

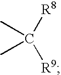

at least one of groups R¹, R², R³, R⁴, R⁵, R⁶ (and R⁸ and R⁹ if present) is the group -E-F where E is a spacer group and F is a reactive group; one of groups R⁷ is selected from —CN, —Cl, —F, —CF₃ and —C(O)R¹⁰ wherein R¹⁰ is selected from H, C₁-C₆ alkyl and aryl. Preferred group R⁷ is meso-substituted —CN, which confers an unexpected hypsochromic shift of approximately 40 nm in the emission spectrum, when compared with the corresponding unsubstituted analogue.

Other fluorescent dye classes in addition to the cyanine dyes may be selected from the fluoresceins, rhodamines, and derivatives of the bis-pyrromethine boron difluoride dyes, such as 3,3′,5,5′-tetramethyl-2,2′-pyrromethene-1,1′-boron difluoride, sold under the trademark BODIPY™ by Molecular Probes Inc. BODIPY™ analogues are disclosed in U.S. Pat. ent Nos. 4,774,339, 5,187,223, 5,248,782 and 5,274,113 (Haugland and Kang), as well as in the “Handbook of Fluorescent Probes and Research Chemicals”, published by Molecular Probes Inc.

Preferably, the cyanine dyes according to formula (I) or (II) are a matched pair of dyes in which n is different for each dye and is 1 or 2; and X and Y are both >C(CH₃)₂.

Suitably, the reactive group F in the compounds of formula (I) or (II) is a group that can react under suitable conditions with a functional group of a protein component to be labelled, such that the compound covalently binds to and thereby labels the component. Preferably, group F in each dye of the matched set of dyes is reactive with hydroxyl, amino, sulphydryl or carboxyl groups. More preferably, the reactive group F is selected from succinimidyl ester, sulpho-succinimidyl ester and maleimide.

Suitably, in the compounds of formula (I) and (II), spacer group E has from 1 to 20 linked atoms, preferably from 6 to 15 atoms. Preferably, E is the group —(CH₂)ₚ—Q—(CH₂)ᵣ— where Q is selected from: —CH₂— and —CO—NH—, p is 1-5 and r is 0-5.

Particular bifunctional, bis-reactive cyanine dyes suitable for labelling amino groups present in protein components, are those in which X and Y are >C(CH₃)₂; R¹ and R² are the group -E-F where E is —(CH₂)₅— and F is succinimidyl ester or sulpho-succinimidyl ester; R³ and R⁴ are hydrogen; and n is 1 or 2.

Particular monofunctional reactive cyanine dyes suitable for labelling amino groups are those in which X and Y are >C(CH₃)₂; one of R¹ and R² is the group -E-F where E is group —(CH₂)₅— and F is succinimidyl ester or sulpho-succinimidyl ester; remaining R¹ or R² is selected from C₁-C₆ alkyl, preferably methyl, ethyl or propyl; R³ and R⁴ are hydrogen; and n is 1 or 2.

Particularly preferred fluorescent cyanine dyes that are especially useful for labelling target proteins with available amino (and hydroxyl) functional groups in proteins are the mono- and bis-reactive N-hydroxysuccinimidyl esters of CY™3 and CY™5. Particularly preferred fluorescent cyanine dyes that are especially useful for labelling target proteins with available sulphydryl functional groups in proteins are the mono- and bis-reactive maleimido derivatives of CY™3 and CY™5.

Examples of matched pairs of dyes according to the general formula (I) are as follows: Set 1, (1a) and (1b) and Set 2, (2a) and (2b):

| | |
|---|---|
| CY ™5 | CY ™3 |

Set 1

| | |
|---|---|
| 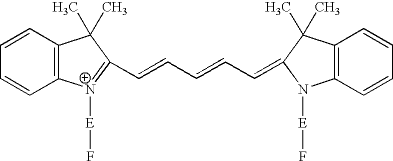 | 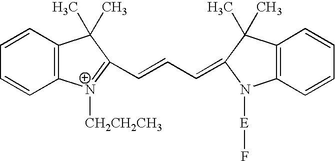 |
| (Compound 1a) | (Compound 1b) |

Set 2

| | |
|---|---|
| 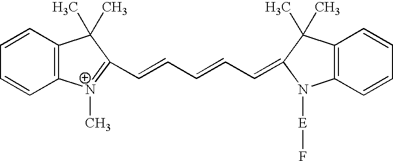 | 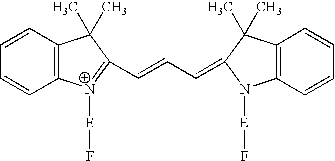 |
| (Compound 2a) | (Compound 2b) |

wherein E is a spacer group as hereinbefore defined and F is a reactive group, preferably selected from succinimidyl ester, sulpho-succinimidyl ester and maleimide.

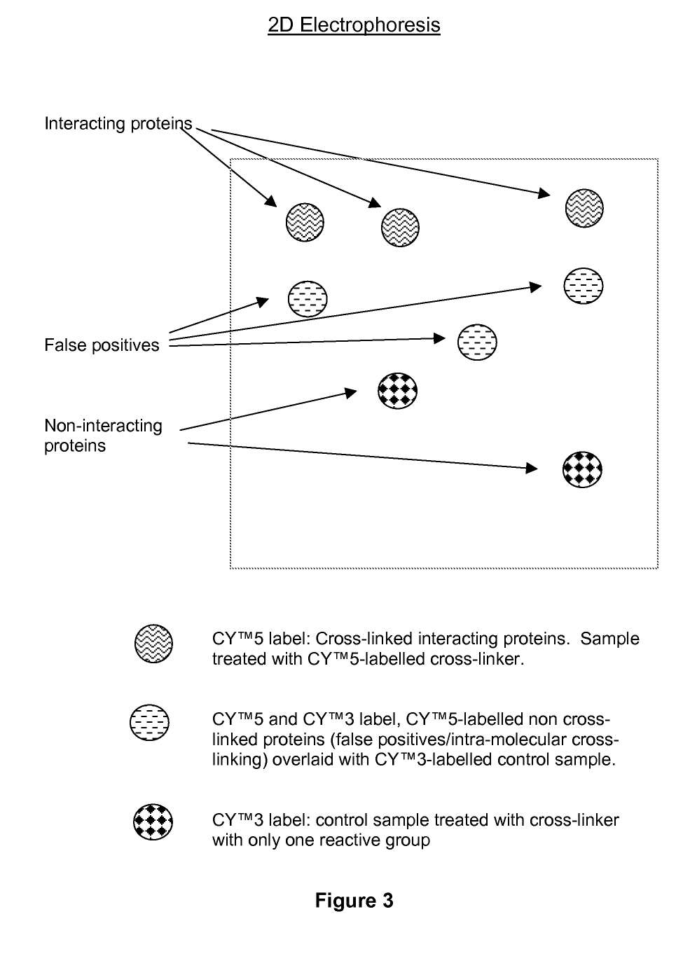

For detecting differences between the test sample and the control sample, suitably the test and control samples are subjected to 1D or 2D electrophoresis, both samples being run in the same gel. In one embodiment (shown in FIG. 3), a CY™5 cross-linking reagent (for example Compound (1a)) is used, and the interacting proteins are identified as spots labelled with CY™5. Non-interacting proteins within the cell will not be labelled with the CY™5 reagent. The control sample, which is reacted with a monofunctional reactive moiety derivatised with a CY™3 (for example Compound (1b)) and therefore unable to cross-link interacting proteins, is run in the same gel. Cross-linked interacting proteins that are labelled with CY™5 will exhibit an altered position in the gel compared with the control, non cross-linked proteins labelled with CY™3.

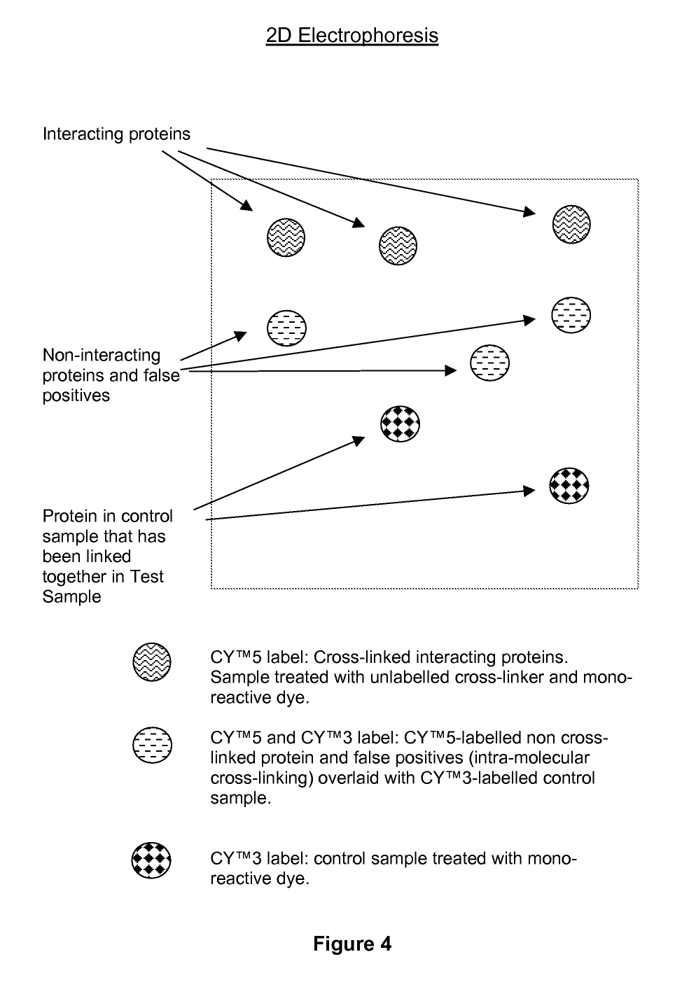

FIG. 4 illustrates a typical spot pattern obtained wherein, as described in the second aspect of the invention, interacting proteins are cross-linked using unlabelled cross-linker, followed by labelling with a luminescent dye selected from a matched set of dyes, for example CY™5 monoreactive dye. As described above, a control sample of lysed cells is labelled with a CY™3 monoreactive dye. The newly appearing CY™5-labelled spots that have an altered position (compared to the CY™3 labelled control sample) are cross-linked interacting proteins that can be identified using standard procedures. The CY™3 and CY™5 double-labelled spots are overlapping spots of non-interacting proteins and false positives from control and test samples. The CY™3 labelled spots represent proteins from the control sample, that in the cross-linked sample are interacting proteins. These spots will be devoid of CY™5 label, since the protein was cross-linked with another protein which leads to an altered position in the gel, due to altered pl and size upon cross-linking.

The reaction with cross-linkers in vivo may be optimised in such a way that only two interacting proteins are cross-linked. A protein-protein interaction map may be assembled using the data obtained for several sets of two interacting proteins. For example, one protein may interact with several different proteins, or alternatively, several proteins may together form a complex. The internal organisation of such a complex may be determined from the collected analytical data from the cross-linked proteins. The identification of false positives (in which a cross-linking group is attached to single protein (via intra-molecular cross-linking) and cross-linked spots overlapping with non cross-linked spots is avoided by applying a control sample to each gel.

The method of the present invention may be applied in the detection of intermolecular association between different proteins in a cell sample, wherein a non cross-linked control sample is labelled with CY™2 monofunctional reagent (for example, a cross-linker with only one reactive group) and two different cross-linked samples are labelled respectively with a CY™3 and CY™5 (labelled or unlabelled cross-linker). Multiplexing of samples in differential protein analysis has greatly improved the detection of protein levels between different samples. However, in order to avoid incorrect biological conclusions resulting from the interpretation of gels, the method of the present invention employs a control sample which is included on each gel and is run with each separation. The use of an internal standard for detecting differences in protein-protein association effectively removes the system (gel-to-gel) variation, enabling accurate identification of biological change between samples.

By employing cross-linking groups that target specific functional groups on a protein, cross-linking groups may be employed that can target subsets of protein components, such as post-translationally modified proteins, for example phospho-proteins. Alternatively, the protein components may comprise a carbohydrate derivative, or a lipid derivative.

The method according to the present invention may be used with a variety of cell types, including all normal and transformed cells derived from any recognised source with respect to species (e.g. human, rodent, simian), tissue source (e.g. brain, liver, lung, heart, kidney skin, muscle) and cell type (e.g. epithelial, endothelial). There are established protocols available for the culture of diverse cell types. (See for example, Freshney, R. I., Culture of Animal Cells: A Manual of Basic Technique, 2^{nd} Edition, Alan R. Liss Inc. 1987). In addition, samples for use in the method of the invention may be derived from cells which have been transfected with recombinant genes and thereafter cultured; or cells which have been subjected to an external stimulus (such as heat shock or drug treatment).

Intact cells or sub-cellular fractions of cells or tissue are labelled according to the first or second aspects of the invention by reacting test and control samples with bifunctional reactive dyes or monofunctional reactive dyes as described above. For labelling a test sample of intact cells, the cell culture media is removed followed by washing the cells in physiological salt buffer, such as PBS pH 7.4. For adherent cell types, cells are detached from the surface upon which they are growing by non-enzymatic means and re-suspended in a solution suitable for labelling with the above compounds, for example HBSS pH 8.5, 1M urea. For labelling a test sample of sub-cellular fractions of cells or tissue, the sub-cellular fraction is first isolated according to methods previously described for the different types of sub-cellular fractions (see for example isolation of microsomal fraction from fibroblasts, Hannesson et al., Biosynthesis of dermatan sulphate, Biochem. J., (1996), 313, 589-596), followed by re-suspension in a solution suitable for labelling with the above compounds, for example HBSS pH 8.5, 1M urea. The labelling buffer should not contain additives that may disrupt the general native structures of the cell or the sub-cellular fraction. For labelling a control sample of intact cells or sub-cellular fractions, protease inhibitors, such as phenylmethane-sulphonyl fluoride (PMSF), ethylenediaminetetraacetic acid (EDTA), leupeptin, aprotinin, may also be added to the labelling buffer to minimise degradation by endogenous proteases.

Suitably, protein components from the test samples, including dye-labelled proteins, may be isolated from intact cells to form extracts using a variety of well known methods and extraction reagents. Typically, cells from the tissue/culture are disrupted, for example by homogenisation, sonication, cell lysis, and the protein extracted and solubilised in the presence of reagents including denaturing reagents, such as urea, thiourea, detergents such as SDS, CHAPS, TRITON™ X-100, NP-40, reducing agents, such as dithiothreitol (DTT), mercaptoethanol, and buffer such as Tris, HEPES. A number of factors should be considered when selecting the buffer and the pH for extraction, including the degree of buffering capacity required, the influence of temperature and ionic strength on pH, interaction with metal ions and compatibility with subsequent purification procedures. Typically, extraction may be performed in the range pH 5-9. Protease inhibitors, such as phenylmethane-sulphonyl fluoride (PMSF), ethylenediaminetetraacetic acid (EDTA), leupeptin, aprotinin, may also be added to minimise degradation by endogenous proteases.

The dye labelled protein components from the test and control samples are separated by a separation method capable of resolving the samples into discrete components. Techniques for separating cellular proteins and their derivatives are well known. The separation step is typically based on physical properties of the labelled components (e.g. charge and molecular weight) and employs an electrophoretic method, for example, one-dimensional electrophoresis, two-dimensional electrophoresis, capillary zone electrophoresis, capillary gel electrophoresis or isoelectric focussing. A preferred analytical method for separating the labelled proteins, is 2-D gel electrophoresis.

The separated proteins may be detected and/or quantitated by optical means, suitably fluorescence microscopy employing an imaging instrument, such as a CCD camera, fluorescence scanner or confocal imager. By analysing the fluorescent signals emitted from different labelled components, differences in the composition of proteins present between test and control samples may be determined. Comparison of the relative fluorescent signals between different interacting and non-interacting proteins may be used to quantify changes in protein association as a result of induced biological change, for example, as a result of disease or drug treatment. Analysis of gel images is typically performed using software designed specifically for 2-DE analysis. For example, DECYDER™ (GE Healthcare) is fully automated image analysis software for spot detection, background subtraction, normalisation, quantitation, positional matching and differential protein expression analysis of multiplexed images. This enables co-detection of differently labelled samples within the same gel.

Following analysis of the gels using appropriate software, the CY™3-labelled cross-linked protein spots can be picked and identified using standard procedures. Typically, the spots of interest are picked from the gel, proteins are eluted from the gel plug in a suitable solvent. The isolated proteins are digested with a suitable enzyme, for example, trypsin, Lys-C, and the peptide pattern determined by mass-spectroscopy, for example electrospray, or MALDI-MS. The identity of individual protein components may also be determined using MS/MS peptide sequencing. In order to unambiguously identify labelled proteins (and thus interacting proteins), dye-labelled components may be enriched, or purified from unlabelled components, prior to separation and/or MS analysis using a variety of well known methods, for example by the use of solid phase antibodies that bind to the dyes.

CY™, DECYDER™, TYPHOON ™ and IMAGEQUANT™ are trademarks of GE Healthcare.

### EXAMPLES

Below the present invention will be disclosed by way of examples, which are intended solely for illustrative purposes and should not be construed as limiting the present invention as defined in the appended claims. All references mentioned below or elsewhere in the present application are hereby included by reference.
1. Labelling Procedure

Proteins were labelled with either CY™3 mono-reactive NHS ester (GE Healthcare, Product code no. RPK0273) or CY™5 bis-reactive NHS ester dyes (GE Healthcare, Product Code No. PA35000). The model proteins chosen were antigen and antibody pairs known to interact and be closely associated under physiological conditions. Two antibody-protein pairs were tested: the first pair was bovine cardiac muscle actin (Sigma, Product code no. A3653) and mouse anti-actin IgG (clone AC-40, Sigma, Product code no. A4700) and the second pair was transferrin from human plasma (Calbiochem, Product no. 616395) and rabbit anti-transferrin IgG (DakoCytomation, Product code no. A0061).

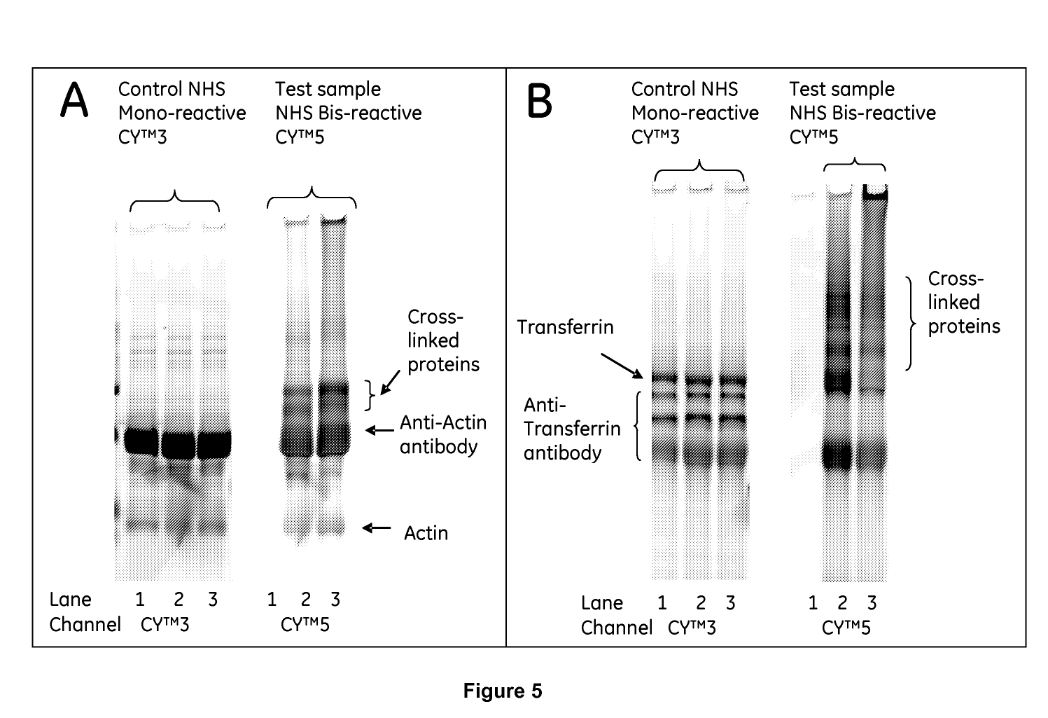

25 μg of each protein were mixed in PBS pH 8.0. One aliquot of the mixture was labelled with 400 pmoles of CY™3 mono-reactive NHS ester and approximately 5 (lane 2, FIG. 5) or 10 (lane 3, FIG. 5) times the recommended amount of bis-reactive NHS CY™5 Dye to protein ratio in the reaction. The CYDYE™ labelled components of the test and control sample were mixed and separated by 1-D SDS PAGE electrophoresis.
2. Protein Separation by 1-D Electrophoresis

1-D electrophoresis was performed using NOVEX® ris-Glycine gels (Invitrogen, Product code no. EC60055BOX), MiniVE vertical Electrophoresis system (GE Healthcare, Product code no. 80-6418-77), EPS 301 Power supply (GE Healthcare, Product code no. 18-1130-01) and PLUSONE™ reagents (GE Healthcare). Samples were mixed with 2×sample loading buffer (120 mM tris pH 6.8, 20% glycerol, 4% SDS, 200 mM DTT and a trace amount of bromophenol blue), heated at 96° C. for 5 minutes, and applied to the gel and run for approximately 2 hours at 100V.
3. Fluorescence Gel Imaging

Labelled proteins were visualised using the TYPHOON™ Imager 9410 (GE Healthcare) with the following settings:

| Channel | Excitation | Emission |
|---|---|---|
| CY ™3 | 540 nm (25 nmBP) | 590 nm (35 nmBP) |
| CY ™5 | 620 nm (30 nmBP) | 680 nm (30 nmBP) |

Exposure times were optimised for individual experiments to give a maximum pixel value on the image of 100,000 to avoid saturation of the signal.
4. Image Analysis

IMAGEQUANT™ 5.2 software (GE Healthcare) was used for analysis of the labelled proteins. The amount of cross-linked proteins in the test sample, detected as newly appearing protein bands with increased molecular weight compared to the control sample was estimated. False positives, as well as cross-linked proteins are shown in FIG. 5.
5. Results

The following results are shown in FIG. 5.

Panel A, lanes 1-3: CY™3 channel shows a control sample of actin—anti-actin antibody labelled with monofunctional reactive CY™3 devoid of any cross-linked proteins.
Panel A, lane 1: CY™5 channel shows no CY™5 signal (no bifunctional reactive CY™5 was loaded in this lane, only CY™3 control labelled sample).
Panel A, lane 2: CY™5 channel shows a test sample of actin and anti-actin antibody labelled with 5 times the recommended amount of bifunctional reactive CY™5 and cross-linked proteins are indicated.
Panel A, lane 3: CY™5 channel shows a test sample of actin and anti-actin antibody labelled with 10 times the recommended amount of bifunctional reactive CY™5 and cross-linked proteins are indicated.
Panel B, lanes 1-3: CY™3 channel shows a control sample of transferrin ant anti-transferrin antibody labelled with monofunctional reactive CY™3 devoid of any cross-linked proteins.
Panel B, lane 1: CY™5 channel shows no CY™5 signal (no bifunctional reactive CY™5 was loaded in this lane, only CY™3 control labelled sample).
Panel B, lane 2: CY™5 channel shows a test sample of transferrin and anti-transferrin antibody labelled with 5 times the recommended amount of bifunctional reactive CY™5 and cross-linked proteins are indicated.
Panel B, lane 3: CY™5 channel shows a test sample of transferrin and anti-transferrin antibody labelled with 10 times the recommended amount of bifunctional reactive CY™5 and cross-linked proteins are indicated.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method of detecting intermolecular association between two components present in a sample, said method comprising:
a) contacting a first aliquot from said sample with a bifunctional reactive moiety under conditions so as to covalently bind to and thereby cross-link said components and wherein said bifunctional reactive moiety comprises a first dye chosen from a matched set of dyes and wherein each dye in said matched set emits luminescent light having a property that is distinguishably different from the emitted luminescent light of the remaining dyes in said matched set;
b) preparing an extract of dye-labelled components from said first aliquot;
c) separating the different dye-labelled components by an electrophoretic method; and
d) detecting differences in a luminescence property between the separated dye-labelled components by luminescence detection;
wherein said separating step c) is performed in the presence of a control comprising an extract of components from a second aliquot from said sample and wherein said second aliquot is contacted with a different dye chosen from said matched set of dyes so as to covalently bind to said components.

2. A method of detecting intermolecular association between two components present in a sample, said method comprising:
a) contacting a first aliquot from said sample with a bifunctional reactive moiety under conditions so as to covalently bind to and cross-link said components;
b) preparing an extract of components from said first aliquot;
c) contacting said extract with a dye chosen from a matched set of dyes wherein each dye in said matched set is capable of selectively labelling said components and wherein each dye emits luminescent light having a property that is distinguishably different from the emitted luminescent light of the remaining dyes in said matched set;
d) separating the different dye-labelled components by an electrophoretic method; and
e) detecting differences in a luminescence property between the separated dye-labelled components by luminescence detection;
wherein said separating step d) is performed in the presence of a control comprising an extract of components from a second aliquot from said sample and wherein said second aliquot is contacted with a different dye chosen from said matched set of dyes so as to covalently bind to said components.

3. The method of claim 1 or claim 2, wherein said components are protein components present in said sample.

4. The method of claim 1 or claim 2, wherein said sample is a cell sample.

5. The method of claim 3, wherein said bifunctional reactive moiety is a homo-bifunctional cross-linking reagent.

6. The method of claim 5, wherein said bifunctional reactive moiety covalently binds to lysine residues in said protein components.

7. The method of claim 5, wherein said bifunctional reactive moiety covalently binds to sulphydryl residues in said protein components.

8. The method of claim 5, wherein said bifunctional reactive moiety covalently binds to carboxyl residues in said protein components.

9. The method of claim 1 or claim 2, wherein said bifunctional reactive moiety is a hetero-bifunctional cross-linking reagent.

10. The method of claim 1 or claim 2, wherein each of said dyes is matched one with the other by virtue of its charge and molecular weight characteristics.

11. The method of claim 1 or claim 2, wherein said matched set of dyes are selected from fluorescent dyes.

12. The method of claim 11, wherein said matched set of dyes are selected from the group consisting of fluoresceins, rhodamines and cyanine dyes.

13. The method of claim 10, wherein said matched set of dyes are selected from cyanine dyes having the structure: 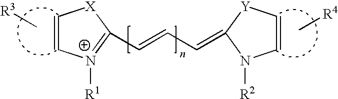
wherein:
the dotted lines each represent carbon atoms necessary for the formation of said dye and are selected independently from phenyl and naphthyl;
X and Y are the same or different and are selected from: >C(CH₃)₂, O and S;
at least one of groups R¹, R², R³ and R⁴ is the group -E-F where E is a spacer group having a chain from 1-20 linked atoms selected from linear or branched C₁₋₂₀ alkyl chains, which may optionally contain one or more linkages selected from —O—, —NR′—, —C(O)—NR′—, —CR′═CR′— and phenyl; where R′ is hydrogen or C₁-C₄ alkyl, and F is a reactive group capable of reacting with a complementary functional group on the protein component to be labelled;
any remaining group R¹ and R² is selected from C₁-C₁₀ alkyl; and
any remaining R³ and R⁴ are selected from hydrogen, sulphonate and sulphonic acid; and
n is an integer from 1 to 3.

14. The method of claim 13, wherein each dye of said set has a target bonding group F which is a reactive group reactive with hydroxyl, amino, sulphydryl or carboxyl groups.

15. The method of claim 13, wherein said reactive group is selected from the group consisting of succinimidyl ester, sulpho-succinimidyl ester and maleimide.

16. The method of claim 11, wherein said matched set of dyes are derivatives of dipyrromethine boron difluoride dyes.

17. The method of claim 11, wherein each of said fluorescent dyes is distinguishable one from the other by virtue of its fluorescence emission wavelength and/or its fluorescence lifetime.

18. The method of claim 3, wherein said protein components include phospho-proteins.

19. The method of claim 1 or claim 2, wherein said components comprise a carbohydrate derivative.

20. The method of claim 1 or claim 2, wherein said components comprise a lipid derivative.

21. The method of claim 1 or claim 2, wherein said electrophoretic method comprises one-dimensional electrophoresis, two-dimensional electrophoresis, capillary zone electrophoresis, capillary gel electrophoresis or isoelectric focussing.

22. The method of claim 1 or claim 2, wherein the step of detecting differences in a luminescence property is by fluorescence microscopy.

23. The method of claim 1 or claim 2, wherein the step of detecting differences in a luminescence property is by optical imaging.
